(19) United States
(12) Patent Application Publication
(10) Pub. No.: US 2024/0175683 A1
(43) Pub. Date: May 30, 2024

(54) ADJUSTMENT OF FREQUENCY OF A MOTION TRACKING SYSTEM

(71) Applicant: Sword Health, S.A., Porto (PT)

(72) Inventors: Pedro Miguel Simoes Bastos Martins, Porto (PT); Pedro Miguel Moreira de Sousa, Porto (PT); Jose Carlos Coelho Alves, Porto (PT); João Paulo Dias Andrade; Mârcio Filipe Moutinho Colunas; Luìs AntÓnio Correia de Oliverira, Porto (PT); Virgìlio AntÓnio Ferro Bento, Porto (PT)

(21) Appl. No.: 18/389,583

(22) Filed: Nov. 14, 2023

(30) Foreign Application Priority Data
Nov. 25, 2022 (EP) 22398024.4

(51) Int. Cl. G01C 21/16; A61B 5/11; G06F 3/0346

(52) U.S. Cl. CPC G01C 21/16; A61B 5/1114; G06F 3/0346; A61B 5/7475; A61B 2562/0219; A61B 2562/04

(57) ABSTRACT

A motion tracking system comprises a computing device and a plurality of motion trackers in communication with the computing device. A user interface provided by the computing device is used to instruct a subject to perform a movement of one or more body members. One or more frequencies of one or more of the plurality of motion trackers is adjusted to reduce a power consumption of at least one of the plurality of motion tracker to thereby reduce a power consumption of the motion tracking system. The one or more frequencies is adjusted based at least in part on the movement which the subject is instructed to perform. The subject is tracked using the plurality of motion trackers of the motion tracking system.

### CLAIM OF PRIORITY

This application claims the benefit of priority to European Patent Application No. 22398024.4, filed on Nov. 25, 2022, which is incorporated herein by reference in its entirety.

### TECHNICAL FIELD

The present disclosure relates to the technical field of motion tracking systems.

### BACKGROUND

Many motion tracking systems have motion trackers, also referred to herein as trackers, that are arranged on a target whose motion is to be tracked. The trackers may comprise one or more inertial sensors which may be part of inertial measurement units. Each inertial sensor is configured for measuring, for example, orientations, angular velocities, accelerations, or forces. These measurements may then be processed by a computing device to derive a motion of the tracked target.

### BRIEF DESCRIPTION OF THE DRAWINGS

To complete the description and in order to provide for a better understanding of the disclosure, a set of drawings is provided. Said drawings form an integral part of the description and illustrate examples of the disclosure, which should not be interpreted as restricting the scope of the disclosure, but just as examples of how the disclosure can be carried out. The drawings comprise the following figures:

FIG. 1 diagrammatically shows a motion tracking system in accordance with some examples.

FIG. 2 shows a person performing predetermined movements while wearing motion trackers, according to some examples.

FIG. 3 shows a person performing predetermined movements while wearing motion trackers, according to some examples.

### DETAILED DESCRIPTION

In order to enhance the accuracy with which a motion tracking system tracks the motion of a target, it may be advantageous to configure the motion tracking system so that measurements are taken and processed at a relatively high frequency. However, using a relatively high frequency causes drawbacks such as a relatively high power consumption and increased data traffic.

The motion trackers may obtain power from, for example, a battery. Therefore, the relatively high consumption of power by the motion trackers causes an increase in the power obtained from the battery and an increase in the number of charge/discharge cycles of the battery, thereby potentially worsening, among others, durability and performance of the battery.

A motion tracking system configured for sampling at a relatively low frequency could decrease the accuracy with which the motion tracking system tracks a motion of the target. Examples described herein may enhance or maintain accuracy of estimations provided by the motion tracking system, such as accuracy of motion tracking, while minimizing a negative impact on communications and power consumption of the motion tracking system. Examples described herein may reduce a total power consumption of the motion tracking system.

In a first aspect of the disclosure, a method for adjusting one or more frequencies of one or more motion trackers of a plurality of motion trackers of a motion tracking system is provided. Each motion tracker of the plurality of motion trackers may be arrangeable on a body member of a user for motion tracking thereof and comprise at least one sensor, the at least one sensor comprising one or more motion sensors configured to measure motion of the one or more motion sensors.

The method may comprise configuring, by each of the one or more motion trackers, at least one frequency associated with the respective motion tracker at least based on a predetermined movement to be performed by the user, thereby modifying a value of at least one predetermined frequency of the respective motion tracker. The at least one predetermined frequency of the respective motion tracker may comprise at least one of: a predetermined measurement frequency with which one or more sensors of the at least one sensor of the respective motion tracker measure a respective physical magnitude, a predetermined digitizing frequency with which the respective tracker digitizes measurements, a predetermined processing frequency with which the respective tracker processes the digitized measurements and a predetermined transmission frequency with which the respective tracker transmits measurements to a computing device of the motion tracking system. The computing device may be configured to receive measurements from the plurality of motion trackers.

The at least one frequency to be configured may comprise at least one of: a measurement frequency, a digitizing frequency, a processing frequency and a transmission frequency.

In some examples, the motion tracking system comprises data indicative of a first plurality of adjustment values for configuring the respective at least one frequency based on both a predetermined movement to be performed by the user and a body member of the user where the respective tracker is arranged, each adjustment value of the first plurality of adjustment values being associated with one predetermined movement to be performed by the user and one body member where one of the motion trackers of the one or more of motion trackers must be arranged, and each adjustment value of the first plurality of adjustment values being configured to be applied to the respective predetermined frequency of the motion tracker.

Accordingly, in some examples, at least one of the measurement frequency, the digitizing frequency, the processing frequency and the transmission frequency associated with the motion tracker is configured based on adjustment values of the first plurality of adjustment values wherein each adjustment value of the adjustment values is associated with one predetermined movement to be performed by the user and one body member where the motion tracker must be arranged.

In some examples, each of the at least one frequency has a corresponding predetermined frequency. The method may configure the at least one frequency associated with the respective motion tracker based on the predetermined movement to be performed by the user and the body member where the respective motion tracker must be arranged. Thereby the method may allow for increasing and decreasing of the at least one frequency for enhancing a tradeoff among high accuracy of the motion tracking and/or of other potential estimations based on measurements of the at least one sensor (e.g. an estimation of temperature and/or respiration rate and/or pulse rate), low power consumption by the respective tracker and, if the transmission frequency is adjusted, low congestion of a channel through which the respective tracker transmits, to the computing device, measurements taken by the respective tracker. In some examples, the method may allow for detecting dynamic movements, such as running and jumping.

In some examples, the at least one frequency comprises the measurement frequency and/or the digitizing frequency, the transmission frequency being the frequency with which the respective tracker transmits at least one of: the measurements taken by the one or more sensors at the measurement frequency, the digitized measurements digitized at the digitizing frequency, and the processed measurements which have been processed at the processing frequency. Thereby, the method may allow adjusting frequencies involved from the acquisition of a measurement by a sensor up to and including the transmission.

In some examples, each motion tracker of the plurality of motion trackers is arrangeable on the body member of the user for motion tracking of the user or for motion tracking of the body member of the user. In some examples, the one or more motion sensors comprise one or more inertial sensors, and each motion sensor is configured to measure a physical magnitude indicative of a motion of the motion sensor. Examples of the one or more inertial sensors are at least one of: an accelerometer, a gyroscope, and a magnetometer. In some examples, the respective motion tracker comprises an inertial measurement unit, the inertial measurement unit comprising the one or more inertial sensors.

In some examples, the at least one predetermined frequency comprises at least one of: a predetermined measurement frequency with which one or more motion sensors of the at least one sensor of the respective motion tracker measure a respective physical magnitude, a predetermined digitizing frequency with which the respective tracker digitizes motion measurements, a predetermined processing frequency with which the respective tracker processes the digitized measurements and a predetermined transmission frequency with which the respective tracker transmits, to the computing device, motion measurements taken by one or more motion sensors of the at least one sensor of the respective tracker at the measurement frequency and/or the digitized measurements digitized at the digitizing frequency and/or the processed measurements which have been processed at the processing frequency. Thereby, the method may allow for increasing and decreasing of the at least one frequency for enhancing a tradeoff among high accuracy of the motion tracking, low power consumption by the motion tracking system and, if the transmission frequency is adjusted, low congestion of a channel through which the respective tracker transmits, to the computing device, the motion measurements.

In some examples, the at least one sensor of the respective motion tracker further comprises one or more sensors configured to measure a physical parameter different from motion, for example: a sensor configured to measure a vital sign of the user, such as, respiration rate of the user, pulse rate of the user, or temperature of the body member of the user where the tracker is arranged.

Since vital signs, such as the respiration rate of the user and the pulse of the user, depend on the predetermined movement performed by the user, the at least one frequency associated with the respective tracker may be configured based on the predetermined movement to be performed by the user. For example, a first predetermined movement which causes a relatively high increase in the respiration rate and/or in the pulse rate of the user may be associated with a first adjustment value being different from a second adjustment value associated with a second predetermined movement which causes a relatively lower increase in the respiration rate and/or in the pulse rate of the user. The first and the second adjustment values may be values of the first plurality of adjustment values, the first adjustment value being associated with the first predetermined movement and the second adjustment value being associated with the second predetermined movement.

In some examples, accuracy of measurements taken by sensors configured to measure a physical parameter different from motion, such as temperature sensors configured to measure a temperature of the body member where the tracker is arranged, may decrease due to a movement of the sensor caused by the performance of the predetermined movement. Therefore, it may be desirable to configure the at least one frequency based on the movement of the sensor, and hence based on the predetermined movement to be performed by the user. For example, a first predetermined movement which causes a relatively high level of motion of a tracker may be associated with a first adjustment value being different from a second adjustment value associated with a second predetermined movement which causes a relatively lower level of motion of the tracker, the first and the second adjustment values being values of the first plurality of adjustment values, the first adjustment value being associated with the first predetermined movement and the second adjustment value being associated with the second predetermined movement. For example, in a second predetermined movement in which a body chest has a higher level of motion than a leg, a third adjustment value is different from a fourth adjustment value, the third and the fourth adjustment values being values of the first plurality of adjustment values, the third adjustment value being associated with the second predetermined movement and with the chest, and the fourth adjustment value being associated with the second predetermined movement and with the leg.

Each adjustment value of the first plurality of adjustment values may be applied to the respective predetermined frequency by, for example, performing an operation (e.g., a sum, a subtraction, a multiplication, or a division) of the adjustment value and the respective predetermined frequency.

In some examples, a first adjustment value of the first plurality of adjustment values is associated with a first predetermined movement and with a first body member having a first tracker arranged thereon, and a second adjustment value of the first plurality of adjustment values is associated with the first predetermined movement and with a second body member having a second tracker arranged thereon. The first predetermined movement may define a movement of the first tracker having a higher level of motion than a movement of the second tracker defined by the first predetermined movement, and the first adjustment value may be such that the application of the first adjustment value to the respective predetermined frequency of the first tracker provides a higher frequency than a frequency provided by the application of the second adjustment value to the respective predetermined frequency of the second tracker. Thereby, the method may allow for increasing accuracy of motion tracking of trackers arranged in body members which should move at a relatively high level of motion according to the predetermined movement.

In some examples, the respective predetermined frequency of the measurement frequency of a tracker of the one or more motion trackers is the predetermined measurement frequency of the tracker. In other words, adjustment of the measurement frequency of the tracker may comprise applying an adjustment value of the first plurality of adjustment values to the predetermined measurement frequency of the tracker. In examples in which the at least one frequency to be configured comprises the measurement frequency of the respective tracker, the at least one predetermined frequency of the respective tracker comprises the predetermined measurement frequency of the respective tracker. The unit of the measurement frequency and of the predetermined measurement frequency may be: number of taken measurements per unit of time, the unit of time being, for example, seconds. Adjusting the measurement frequency may allow for enhancing a tradeoff between low power consumption by the respective tracker and taking measurements at a frequency which is suitable for achieving a certain accuracy of the motion tracking and/or of other potential estimations based on measurements of the at least one sensor (e.g., an estimation of temperature and/or respiration rate and/or pulse rate).

In some examples, the respective predetermined frequency of the digitizing frequency of a tracker of the one or more motion trackers is the predetermined digitizing frequency of the tracker. In other words, adjustment of the digitizing frequency of the tracker may comprise applying an adjustment value of the first plurality of adjustment values to the predetermined digitizing frequency of the tracker. In examples in which the at least one frequency to be configured comprises the digitizing frequency of the respective tracker, the at least one predetermined frequency of the respective tracker may comprise the predetermined digitizing frequency of the respective tracker. The unit of the digitizing frequency and of the predetermined digitizing frequency may be: number of digitizations of measurements per unit of time, the unit of time being, for example, seconds. Adjusting the digitizing frequency may allow for enhancing a tradeoff between low power consumption by the respective tracker and providing digitized measurements at a frequency which is suitable for achieving a certain accuracy of the motion tracking and/or of other potential estimations based on measurements of the at least one sensor (e.g., an estimation of temperature and/or respiration rate and/or pulse rate).

In some examples, the measurements digitized by the respective tracker are the measurements taken by the one or more sensors of the at least one sensor of the respective tracker at the measurement frequency. For example, the one or more sensors may provide measurements of acceleration at a measurement frequency of 100 Hz by providing one output voltage proportional to the acceleration every 10 milliseconds, the output voltages being digitized by the respective tracker.

In some examples, the measurements digitized by the respective tracker are measurements taken by one or more sensors of the at least one sensor of the respective tracker, the measurements being continuously taken over time. For example, the one or more sensors of the respective tracker may be configured to measure a physical magnitude in a continuous manner over time and the resulting measurements in some particular instants of time may be digitized by the respective tracker at the digitizing frequency.

The respective predetermined frequency of the processing frequency of a tracker of the one or more motion trackers may be the predetermined processing frequency of the tracker. In other words, adjustment of the processing frequency of the tracker may comprise applying an adjustment value of the first plurality of adjustment values to the predetermined processing frequency of the tracker. In examples in which the at least one frequency to be configured comprises the processing frequency of the respective tracker, the at least one predetermined frequency of the respective tracker comprises the predetermined processing frequency of the respective tracker. The unit of the processing frequency and of the predetermined processing frequency may be: number of processed measurements per unit of time, the unit of time being, for example, seconds. Adjusting the processing frequency may allow for enhancing a tradeoff between low power consumption by the respective tracker and providing processed measurements at a frequency which is suitable for achieving a certain accuracy of the motion tracking and/or of other potential estimations based on measurements of the at least one sensor (e.g., an estimation of temperature and/or respiration rate and/or pulse rate).

The respective predetermined frequency of the transmission frequency of a tracker of the one or more motion trackers may be the predetermined transmission frequency of the tracker. In other words, adjustment of the transmission frequency of the tracker may comprise applying an adjustment value of the first plurality of adjustment values to the predetermined transmission frequency of the tracker. In examples in which the at least one frequency to be configured comprises the transmission frequency of the respective tracker, the at least one predetermined frequency of the respective tracker comprises the predetermined transmission frequency of the respective tracker. The unit of the transmission frequency and of the predetermined transmission frequency may be: number of transmitted measurements per unit of time, the unit of time being, for example, seconds. Adjusting the transmission frequency may allow for enhancing a tradeoff among transmitting measurements at a frequency which is suitable for achieving a certain accuracy of the motion tracking and/or of other potential estimations based on measurements of the at least one sensor (e.g. an estimation of temperature and/or respiration rate and/or pulse rate), low power consumption by the motion tracking system and low congestion of a channel through which the measurements are transmitted to the computing device.

In some examples, the measurements transmitted by the respective tracker are digital measurements resulting from digitization by the respective tracker.

In some examples, the measurements transmitted by the respective tracker are processed measurements resulting from processing of digital measurements by the respective tracker.

In some examples, at least one of the measurement frequency associated with the respective tracker and the transmission frequency associated with the respective tracker is adjusted. Decreasing the measurement frequency and/or the transmission frequency of a tracker may potentially allow a relatively high reduction of power consumed by the tracker when compared to decreasing the processing frequency of the tracker and/or the digitizing frequency of the tracker. In some cases, this difference of power consumption is particularly high in trackers which have a low processing load.

In some examples, the digitizing frequency associated with the respective tracker and at least one of the measurement frequency associated with the respective tracker and the transmission frequency associated with the respective tracker are adjusted. Decreasing the digitizing frequency and decreasing the measurement frequency and/or the transmission frequency of a tracker may potentially allow for a relatively high reduction of power consumed by the tracker when compared to decreasing the processing frequency. In some cases, this difference of power consumption is particularly high in the trackers which have a low processing load and have a high digitizing frequency.

In some examples, the measurement frequency of the respective tracker is equal to the transmission frequency of the respective tracker. Thereby, measurements are taken and transmitted at the same frequency, so that the transmission frequency has a minimum value allowing transmission of all measurements taken by the one or more sensors of the respective tracker.

In some examples, the digitizing frequency of the respective tracker is equal to the transmission frequency of the respective tracker. Thereby, measurements are digitized and transmitted at the same frequency, so that the transmission frequency has a minimum value allowing transmission of all measurements digitized by the respective tracker at the digitizing frequency or, in other words, so that the digitizing frequency has a minimum value allowing digitization of all measurements subsequently transmitted by the respective tracker at the transmission frequency. Increasing just one of the frequencies, e.g., the digitizing frequency or the transmission frequency, without increasing the other frequency, may increase power consumption by the respective tracker without enabling an increase in accuracy of the motion tracking and/or the other estimations by the motion tracking system. For example, if the digitizing frequency is increased without increasing the transmission frequency, additionally digitized measurements resulting from the increase in the digitizing frequency would not be transmitted, and hence would not be used by the motion tracking system for providing a motion tracking sequence and/or the other estimations.

In some examples, the processing frequency of the respective tracker is equal to the transmission frequency of the respective tracker. Thereby, measurements are processed and transmitted at the same frequency, so that the transmission frequency has a minimum value allowing transmission of all measurements processed by the respective tracker at the processing frequency or, in other words, so that the processing frequency has a minimum value allowing processing of all measurements subsequently transmitted by the respective tracker at the transmission frequency.

In some examples, at least one of the following conditions apply: the transmission frequency is lower than or equal to the processing frequency, the processing frequency is lower than or equal to the digitizing frequency, and the digitizing frequency is lower than or equal to the measurement frequency. Thereby, the method may potentially enable the motion tracking system to not waste (or waste less) energy in increasing frequencies which do not contribute to increasing motion tracking accuracy (e.g., prevents the system from increasing the transmission frequency above the digitizing frequency, thereby avoiding a transmission frequency being higher than required for transmission of all measurements digitized at the digitizing frequency).

In some examples, at least one of the following conditions apply: the transmission frequency is higher than the processing frequency, the processing frequency is higher than the digitizing frequency, and the digitizing frequency is higher than the measurement frequency. These examples may use upsampling wherein, for example, the processing frequency is higher than the measurement frequency and/or the digitizing frequency.

In some examples, the computing device of the motion tracking system is a first computing device and the motion tracking system comprises a second computing device.

In some examples, the second computing device comprises or provides the data indicative of the first plurality of adjustment values.

In some examples, the second computing device is configured to determine an adjustment value of the first plurality of adjustment values based on the data indicative of the first plurality of adjustment values, data indicative of the predetermined movement to be performed by the user, and data indicative of a body member where a tracker is arranged or must be arranged, the adjustment value being configured to be applied to the at least one predetermined frequency of the tracker. In some examples, the second computing device is configured to transmit the determined adjustment value to the tracker and/or to transmit, to the tracker, a result of the application of the determined adjustment value to the respective predetermined frequency of the tracker.

In some examples, the first computing device is configured to provide a motion tracking sequence of the user or of body members of the user based on measurements received from the plurality of motion trackers.

In some examples, the first computing device comprises or provides the data indicative of the first plurality of adjustment values.

In some examples, the first computing device is configured to determine an adjustment value of the first plurality of adjustment values based on the data indicative of the first plurality of adjustment values, data indicative of the predetermined movement to be performed by the user, and data indicative of a body member where a tracker is arranged or must be arranged, the adjustment value being configured to be applied to the at least one predetermined frequency of the tracker. In some examples, the first computing device is configured to transmit the determined adjustment value to the tracker and/or to transmit, to the tracker, a result of the application of the determined adjustment value to the respective predetermined frequency of the tracker.

In some examples, the method further comprises receiving, by each motion tracker of the one or more motion trackers, from the computing device, the data indicative of the first plurality of adjustment values or indicative of an adjustment value of the first plurality of adjustment values for configuring the at least one frequency based on both the predetermined movement to be performed by the user and the body member of the user where the respective motion tracker is arranged. As a result, each motion tracker may receive the data indicative of the first plurality of adjustment values, so that each motion tracker may determine the adjustment value of the first plurality of adjustment values associated with the predetermined movement to be performed by the user and the body member where the respective tracker must be arranged, or each motion tracker may receive the adjustment value associated with the predetermined movement to be performed by the user and the body member where the respective tracker must be arranged.

In some examples, each adjustment value of the first plurality of adjustment values is further associated with the user and a parameter indicative of a level of motion of the respective tracker as measured by the one or more motion sensors of the respective tracker in a previous performance of the predetermined movement. Thereby, each adjustment value of the first plurality of adjustment values may be associated with the user and with characteristics of a previous performance, by the user, of the predetermined movement. In this way, the one or more frequencies may be adjusted to each particular user, such as a user with a particular injury or with a particular disability.

Depending on the physical condition of each user, the execution of the predetermined movement may be faster or slower. For example, a person that suffers a lot of pain when executing the predetermined movement may be likely to execute the movement at a lower level of motion, e.g., with lower intensity, for example, slower and/or with lower accelerations and/or decelerations. Therefore, the association of each adjustment value of the first plurality of adjustment values with one user, enables that the first plurality of adjustment values take into account the physical condition of the user.

In the context of the present disclosure, the expression “level of motion” referred to a tracker, such as “level of motion of the respective tracker,” or “level of motion of the motion tracker,” refers to characteristics of a motion of the tracker, for example, to at least one of: a position of the tracker, an orientation of the tracker, and a relation of a position and/or orientation of the tracker with respect to time. The relation of a position and/or orientation of the tracker with respect to time comprises, among other possible units and types of measurements, at least one of: speed of the tracker, linear speed of the tracker, angular speed of the tracker, acceleration of the tracker, linear acceleration of the tracker, angular acceleration of the tracker, variation of acceleration of the tracker, variation of linear acceleration of the tracker and variation of angular acceleration of the tracker. In some examples, the level of motion may be a level of motion of the tracker defined by a motion of the tracker in an interval of time, the interval having a lower limit a and an upper limit b wherein a<b.

In some examples, the step of configuring at least based on the predetermined movement to be performed by the user is conducted either prior to the user starting to perform the predetermined movement, or while the user performs the predetermined movement. For example, the motion tracking system may receive or generate a signal indicative of the predetermined movement to be performed by the user, such as knee-ups, push-ups, or squats. In some examples, the signal may be generated by a selection, e.g., by the user, of the predetermined movement via a user interface (UI) of the motion tracking system. In some examples, the signal may be generated based on data indicative of movements to be performed by the user and associated with the user. The motion tracking system may adjust the at least one frequency before the user starts performing the predetermined movement or while the user performs the predetermined movement. In some examples, the step of configuring at least based on the predetermined movement to be performed by the user is conducted after the previous performance, by the user, of the predetermined movement and before another performance, by the user, of the predetermined movement.

In some examples, the method further comprises configuring, by each of the one or more motion trackers, the at least one frequency associated with the respective motion tracker at least based on measurements of the motion measurement of the respective motion tracker, thereby modifying a value of the at least one predetermined frequency of the respective motion tracker. The motion tracking system may comprise data indicative of a second plurality of adjustment values for configuring the respective at least one frequency based on measurements of the motion tracker, each adjustment value of the second plurality of adjustment values being associated with a level of motion of the motion tracker as measured by the one or more motion sensors being within a predetermined motion range, and each adjustment value of the second plurality of adjustment values being configured to be applied to the respective predetermined frequency of the motion tracker.

The adjustment of the one or more frequencies may comprise applying, to the respective predetermined frequency, an adjustment value of the first plurality of adjustment values and an adjustment value of the second plurality of adjustment values. In some examples, the application of the adjustment value of the second plurality of adjustment values allows adjusting the one or more frequencies to characteristics of the actual movements performed by the user. For example, a user may perform the predetermined movement in a way such that the respective tracker may be subjected to a higher level of motion (e.g., faster) than it is expected according to the adjustment value of the first plurality of adjustment values associated with the predetermined movement and with the body member where the respective tracker is arranged. The adjustment value of the second plurality of adjustment values associated with the higher level of motion of the respective tracker may be an adjustment value which upon application to the respective at least one predetermined frequency results in the respective at least one frequency having a higher value than a value of the respective at least one frequency resulting from an application of the adjustment value of the first plurality of adjustment values to the respective at least one predetermined frequency. In some examples, increasing the at least one frequency may allow for an increase in the accuracy of the motion tracking at the cost of increasing power consumption by the respective tracker.

In some examples, the measurements of the motion measurement are measurements taken by the one or more motion sensors.

The level of motion may be within the predetermined motion range. The level of motion comprises, for example, estimated positions and/or speeds and/or accelerations of the motion tracker based on measurements taken by the one or more motion sensors of the respective tracker.

In a second aspect of the disclosure, a method for adjusting one or more frequencies of one or more motion trackers of a plurality of motion trackers of a motion tracking system is provided. Each motion tracker of the plurality of motion trackers is arrangeable on a body member of a user for motion tracking thereof and comprising at least one sensor, the at least one sensor comprising one or more motion sensors configured to measure motion of the one or more motion sensors, and the method comprising configuring, by each of the one or more motion trackers, at least one frequency associated with the respective motion tracker based on measurements of the motion measurement of the respective motion tracker, thereby modifying a value of at least one predetermined frequency of the respective motion tracker.

The at least one predetermined frequency of the respective motion tracker may comprise at least one of: a predetermined measurement frequency with which one or more sensors of the at least one sensor of the respective motion tracker measure a respective physical magnitude, a predetermined digitizing frequency with which the respective tracker digitizes measurements, a predetermined processing frequency with which the respective tracker processes the digitized measurements and a predetermined transmission frequency with which the respective tracker transmits measurements to a computing device of the motion tracking system. The computing device may be configured to receive measurements from the plurality of motion trackers. The at least one frequency to be configured may comprise at least one of: a measurement frequency, a digitizing frequency, a processing frequency, and a transmission frequency.

The motion tracking system may comprise data indicative of a second plurality of adjustment values for configuring the respective at least one frequency based on measurements of the motion tracker, each adjustment value of the second plurality of adjustment values being associated with a level of motion of the motion tracker as measured by the one or more motion sensors being within a predetermined motion range, and each adjustment value of the second plurality of adjustment values being configured to be applied to the respective predetermined frequency of the motion tracker.

Accordingly, at least one of the measurement frequency, the digitizing frequency, the processing frequency, and the transmission frequency associated with the motion tracker may be configured based on a second plurality of adjustment values wherein each adjustment value of the second plurality of adjustment values is associated with a level of motion of the motion tracker as measured by the one or more motion sensors of the motion tracker, the level of motion being within a predetermined motion range.

In some examples, the at least one frequency comprises the measurement frequency and/or the digitizing frequency; the transmission frequency being the frequency with which the respective tracker transmits at least one of: the measurements taken by the one or more sensors at the measurement frequency, the digitized measurements digitized at the digitizing frequency and the processed measurements which have been processed at the processing frequency. Thereby, the method allows adjusting frequencies involved since the acquisition of a measurement by a sensor up to and including the transmission.

The use of the second plurality of adjustment values does not require, but is compatible with, the use of the first plurality of adjustment values.

In the second aspect of the disclosure, the at least one frequency may be configured based on a level of motion of the motion tracker as measured by the one or more motion sensors of the tracker.

In some examples, the one or more motion sensors of the respective tracker comprise one or more inertial sensors, and each motion sensor is configured to measure a physical magnitude indicative of a motion of the motion sensor. Examples of the one or more inertial sensors are at least one of: a gyroscope, an accelerometer, and a magnetometer. In some examples, the respective tracker comprises an inertial measurement unit, the inertial measurement unit comprising the one or more inertial sensors.

In some examples, the at least one sensor of the respective motion tracker further comprises, for example: a sensor configured to measure a vital sign of the user, such as respiration rate of the user, pulse rate of the user, or a temperature of the body member of the user where the tracker is arranged.

Since vital signs, such as the respiration rate of the user and the pulse of the user, depend on a level of motion of the user, the at least one frequency associated with the respective tracker may be configured based on a level of motion of the respective tracker as measured by the one or more motion sensors of the respective tracker being within a predetermined motion range. For example, the respective tracker may move at a relatively high level of motion as a result of a fast movement of the body member where the respective tracker is arranged or must be arranged. In addition, the respective tracker may move at a relatively low level of motion as a result of a slow movement of the body member where the respective tracker is arranged. In order to enhance a tradeoff between high accuracy of motion tracking and/or of other potential estimations based on measurements of the at least one sensor (e.g. an estimation of temperature and/or respiration rate and/or pulse rate) and low power consumption by the tracker, the relatively high level of motion of the tracker may be associated with an adjustment value of the second plurality of adjustment values which is higher or lower than an adjustment value of the second plurality of adjustment values associated with the relatively low level of motion of the respective tracker.

Accuracy of measurements taken by other sensors, such as temperature sensors configured to measure a temperature of the body member where the tracker is arranged, may decrease due to a movement of the sensor caused by the performance of a movement. Therefore, it may be desirable to configure the at least one frequency based on the movement of the sensor, and hence based on the level of motion of the tracker. For example, a first movement which is performed at a relatively high level of motion of a tracker, wherein the relatively high level of motion may be associated with a first adjustment value being higher or lower than a second adjustment value associated with a relatively lower level of motion of the tracker at which a second movement is performed, the first and the second adjustment values being values of the second plurality of adjustment values, the first adjustment value being associated with the relatively high level of motion of the tracker and the second adjustment value being associated with the relatively low level of motion of the tracker. This may allow for enhancing a tradeoff between high accuracy of motion tracking and/or of other potential estimations based on measurements of the at least one sensor (e.g., an estimation of temperature and/or respiration rate and/or pulse rate) and low power consumption by the tracker.

Similarly to the first aspect of the disclosure, the second aspect of the disclosure allows increasing and decreasing the at least one frequency for enhancing a tradeoff among high accuracy of the motion tracking and/or of other potential estimations based on measurements of the at least one sensor (e.g. an estimation of temperature and/or respiration rate and/or pulse rate), low power consumption by the respective tracker and, if the transmission frequency is adjusted, low congestion of a channel through which the respective tracker transmits measurements taken by the respective tracker to the computing device. In addition, the second aspect of the disclosure may allow detecting dynamic movements, such as running and jumping.

The level of motion comprises, for example, estimated positions and/or speeds and/or accelerations of the motion tracker based on measurements taken by the one or more motion sensors of the tracker.

Each motion tracker of the plurality of motion trackers may be arrangeable on a body member of the user for motion tracking of the user or for motion tracking of the body member of the user.

In some examples, the one or more trackers comprise the data indicative of the second plurality of adjustment values.

In some examples, each tracker of the one or more trackers is configured to determine an adjustment value of the second plurality of adjustment values based on the data indicative of the second plurality of adjustment values, data indicative of a level of motion of the respective tracker as measured by the one or more motion sensors of the respective tracker, the level of motion being within a predetermined motion range.

In some examples, the computing device of the motion tracking system is a first computing device and the motion tracking system comprises a second computing device.

In some examples, the second computing device comprises or provides the data indicative of the second plurality of adjustment values.

In some examples, the second computing device is configured to determine an adjustment value of the second plurality of adjustment values based on the data indicative of the second plurality of adjustment values, data indicative of a level of motion of a motion tracker as measured by the one or more motion sensors of the tracker, the level of motion being within a predetermined motion range. In some examples, the second computing device is configured to transmit the determined adjustment value to the tracker and/or to transmit a result of the application of the determined adjustment value to the respective predetermined frequency of the tracker.

In some examples, the first computing device is configured to provide a motion tracking sequence of the user or of body members of the user based on measurements received from the plurality of motion trackers.

In some examples, the first computing device comprises the data indicative of the second plurality of adjustment values.

In some examples, the first computing device is configured to determine an adjustment value of the second plurality of adjustment values based on the data indicative of the second plurality of adjustment values, data indicative of a level of motion of a motion tracker as measured by the one or more motion sensors of the tracker, the level of motion being within a predetermined motion range. In some examples, the first computing device is configured to transmit the determined adjustment value to the tracker and/or to transmit a result of the application of the determined adjustment value to the respective predetermined frequency of the tracker to the tracker.

In some examples, a first adjustment value of the second plurality of adjustment values is associated with a first level of motion of a tracker, and a second adjustment value of the second plurality of adjustment values is associated with a second level of motion of the tracker; the first level of motion being higher than the second level of motion; and the first adjustment value being such that the application of the first adjustment value to the respective predetermined frequency of the tracker provides a higher frequency than a frequency provided by the application of the second adjustment value to the respective predetermined frequency of the tracker. As a result, the method may allow for increasing accuracy of motion tracking of trackers which move at a relatively high level of motion.

The adjustment value of the second plurality of adjustment values may be applied to the respective predetermined frequency by, for example, performing an operation (e.g., a sum, a subtraction, a multiplication, or a division) of the adjustment value and the respective predetermined frequency.

The respective predetermined frequency of the measurement frequency of a tracker of the one or more motion trackers may be the predetermined measurement frequency of the tracker. In other words, adjustment of the measurement frequency of the tracker may comprise applying an adjustment value of the second plurality of adjustment values to the predetermined measurement frequency of the tracker. In examples in which the at least one frequency to be configured comprises the measurement frequency of the respective tracker, the at least one predetermined frequency of the respective tracker comprises the predetermined measurement frequency of the respective tracker. The unit of the measurement frequency and of the predetermined measurement frequency may be: number of taken measurements per unit of time, the unit of time being, for example, seconds. Adjusting the measurement frequency may allow for enhancing a tradeoff between low power consumption by the respective tracker and taking measurements at a frequency which is suitable for achieving a certain accuracy of the motion tracking and/or of other potential estimations based on measurements of the at least one sensor (e.g., an estimation of temperature and/or respiration rate and/or pulse rate).

The respective predetermined frequency of the digitizing frequency of a tracker of the one or more motion trackers may be the predetermined digitizing frequency of the tracker. In other words, adjustment of the digitizing frequency of the tracker may comprise applying an adjustment value of the second plurality of adjustment values to the predetermined digitizing frequency of the tracker. In examples in which the at least one frequency to be configured comprises the digitizing frequency of the respective tracker, the at least one predetermined frequency of the respective tracker comprises the predetermined digitizing frequency of the respective tracker. The unit of the digitizing frequency and of the predetermined digitizing frequency may be: number of digitizations of measurements per unit of time, the unit of time being, for example, seconds. Adjusting the digitizing frequency may allow for enhancing a tradeoff between low power consumption by the respective tracker and providing digitized measurements at a frequency which is suitable for achieving a certain accuracy of the motion tracking and/or of other potential estimations based on measurements of the at least one sensor (e.g., an estimation of temperature and/or respiration rate and/or pulse rate).

In some examples, the measurements digitized by the respective tracker are the measurements taken by the at least one sensor of the respective tracker at the measurement frequency.

In some examples, the measurements digitized by the respective tracker are measurements taken by one or more of the at least one sensor of the respective tracker, the measurements being continuously taken over time. For example, the at least one sensor of the respective tracker may be configured to measure a physical magnitude in a continuous manner over time and the resulting measurements in some particular instants of time may be digitized by the respective tracker at the digitizing frequency.

The respective predetermined frequency of the processing frequency of a tracker of the one or more motion trackers may be the predetermined processing frequency of the tracker. In other words, adjustment of the processing frequency of the tracker may comprise applying an adjustment value of the second plurality of adjustment values to the predetermined processing frequency of the tracker. In examples in which the at least one frequency to be configured comprises the processing frequency of the respective tracker, the at least one predetermined frequency of the respective tracker may comprise the predetermined processing frequency of the respective tracker. The unit of the processing frequency and of the predetermined processing frequency may be: number of processed measurements per unit of time, the unit of time being, for example, seconds. Adjusting the processing frequency may allow for enhancing a tradeoff between low power consumption by the respective tracker and providing processed measurements at a frequency which is suitable for achieving a certain accuracy of the motion tracking and/or of other potential estimations based on measurements of the at least one sensor (e.g., an estimation of temperature and/or respiration rate and/or pulse rate).

The respective predetermined frequency of the transmission frequency of a tracker of the one or more motion trackers may be the predetermined transmission frequency of the tracker. In other words, adjustment of the transmission frequency of the tracker may comprise applying an adjustment value of the second plurality of adjustment values to the predetermined transmission frequency of the tracker. In examples in which the at least one frequency to be configured comprises the transmission frequency of the respective tracker, the at least one predetermined frequency of the respective tracker comprises the predetermined transmission frequency of the respective tracker. The unit of the transmission frequency and of the predetermined transmission frequency may be: number of transmitted measurements per unit of time, the unit of time being, for example, seconds. Adjusting the transmission frequency may allow for enhancing a tradeoff among transmitting measurements at a frequency which is suitable for achieving a certain accuracy of motion tracking and/or of other potential estimations based on measurements of the at least one sensor (e.g., an estimation of temperature and/or respiration rate and/or pulse rate), low power consumption by the motion tracking system and low congestion of a channel through which the measurements are transmitted to the computing device.

In some examples, the measurements transmitted by the respective tracker are digital measurements resulting from digitization by the respective tracker.

In some examples, the measurements transmitted by the respective tracker are processed measurements resulting from processing of digital measurements by the respective tracker.

In some examples, at least one of the measurement frequency associated with the respective tracker and the transmission frequency associated with the respective tracker is adjusted. Decreasing the measurement frequency and/or the transmission frequency of a tracker potentially may enable a relatively high reduction of power consumed by the tracker when compared to decreasing the processing frequency of the tracker and the digitizing frequency of the tracker. This difference of power consumption may be particularly high in trackers which have a low processing load.

In some examples, the digitizing frequency associated with the respective tracker and at least one of the measurement frequency associated with the respective tracker and the transmission frequency associated with the respective tracker may be adjusted. Decreasing the digitizing frequency and decreasing the measurement frequency and/or the transmission frequency of a tracker may enable a relatively high reduction of power consumed by the tracker when compared to decreasing the processing frequency and compared to not decreasing the digitizing frequency. This difference of power consumption may be particularly high in trackers which have a low processing load and have a high digitizing frequency.

In some examples, the measurement frequency of the respective tracker is equal to the transmission frequency of the respective tracker. Thereby, measurements are taken and transmitted at the same frequency, so that the transmission frequency has a minimum value allowing transmission of all measurements taken by the one or more sensors of the respective tracker.

In some examples, the digitizing frequency of the respective tracker is equal to the transmission frequency of the respective tracker. Thereby, measurements are digitized and transmitted at the same frequency, so that the transmission frequency has a minimum value allowing transmission of all measurements digitized by the respective tracker at the digitizing frequency or, in other words, so that the digitizing frequency has a minimum value allowing digitization of all measurements subsequently transmitted by the respective tracker at the transmission frequency. Increasing just one of the frequencies, e.g., the digitizing frequency or the transmission frequency, without increasing the other frequency, may increase power consumption by the respective tracker without enabling an increase in accuracy of the motion tracking and/or the other estimations by the motion tracking system. For example, if the digitizing frequency is increased without increasing the transmission frequency, additionally digitized measurements resulting from the increase in the digitizing frequency would not be transmitted and hence would not be used by the motion tracking system for providing a motion tracking sequence and/or other estimations.

In some examples, the processing frequency of the respective tracker is equal to the transmission frequency of the respective tracker. Thereby, measurements are processed and transmitted at the same frequency, so that the transmission frequency has a minimum value allowing transmission of all measurements processed by the respective tracker at the processing frequency or, in other words, so that the processing frequency has a minimum value allowing processing of all measurements subsequently transmitted by the respective tracker at the transmission frequency.

In some examples, at least one of the following conditions apply: the transmission frequency is lower than or equal to the processing frequency, the processing frequency is lower than or equal to the digitizing frequency, and the digitizing frequency is lower than or equal to the measurement frequency. Thereby, the method may enable the motion tracking system to not waste energy (or waste less energy) in increasing frequencies which do not contribute to increasing motion tracking accuracy (e.g., prevents from increasing the transmission frequency above the digitizing frequency, thereby avoiding a transmission frequency being higher than required for transmission of all measurements digitized at the digitizing frequency).

In some examples, at least one of the following conditions apply: the transmission frequency is higher than the processing frequency, the processing frequency is higher than the digitizing frequency, and the digitizing frequency is higher than the measurement frequency. These examples may use upsampling wherein, for example, the processing frequency is higher than the measurement frequency and/or the digitizing frequency.

In some examples, the step of configuring based on the measurements comprises configuring the transmission frequency of the respective motion tracker with an adjustment value of the second plurality of adjustment values corresponding to no transmission when the level of motion of the respective motion tracker is within a predetermined motion range associated with being motionless. This may enable the motion tracking system to reduce or minimize energy wasted on transmission of measurements of body members which are motionless or substantially motionless and decreasing congestion of a channel through which the respective tracker transmits the measurements. The transmission frequency, e.g., the frequency with which the respective tracker transmits the measurements to the computing device of the motion tracking system, may be set to zero. The level of motion comprises, for example, estimated positions and/or speeds and/or accelerations of the tracker, the estimation being based on measurements taken by the one or more motion sensors of the respective tracker. The predetermined range associated with being motionless is, for example, a range of speed or acceleration which comprises the value of speed equal to zero or acceleration equal to zero. The predetermined range associated with being motionless may be based on the body member where the tracker is arranged or must be arranged. The predetermined range associated with being motionless for a first body member may be different to the predetermined range associated with a body member which is not the first body member. For example, the predetermined range associated with a motionless toe may be narrower than the predetermined range associated with a motionless lower leg.

In some examples, the method further comprises transmitting, to the computing device by each motion tracker of the one or more motion trackers whose transmission frequency has been configured with the adjustment value corresponding to no transmission, data indicative of at least temporal no transmission of measurements by the respective motion tracker. In some examples, the method includes, after non reception by the computing device of measurements from a tracker of the one or more motion trackers during a predetermined period of time if data indicative of at least temporal no transmission has not been received from the tracker, processing, by the computing device, of most recent measurements received from the tracker to determine whether a level of motion of the tracker is within the predetermined motion range associated with the tracker being motionless, the computing device halting a motion tracking procedure otherwise.

The method may thus allow detecting, by the computing device, the level of motion of a member of the body where the tracker is arranged, the level of motion being within the predetermined motion range associated with being motionless. In some examples, if the transmission frequency with which the respective tracker transmits the measurements to the computing device has been configured with the adjustment value corresponding to no transmission, the respective motion tracker, e.g., the motion tracker arranged in the body member which is motionless or substantially motionless, transmits data indicative of temporal no transmission of measurements by the respective motion tracker. In other words, the motion tracker transmits data indicative of an absence of transmission of measurements by the respective motion tracker, the absence of transmission of measurements being temporal. Accordingly, an absence of transmission of measurements by the respective motion tracker to the computing device does not necessarily mean an absence of transmission of data different from measurements by the respective tracker, such as the data indicative of at least temporal no transmission of measurements by the respective motion tracker, to the computing device. Accordingly, an absence of transmission of measurements by the respective motion tracker to the computing device may mean, but does not necessarily mean, an absence of transmission of any measurements by the respective tracker. For example, the absence of transmission may be an absence of transmission of motion measurements; enabling transmission of non-motion measurements.

In some examples, the method comprises configuring a measurement frequency with an adjustment value corresponding to measuring and/or the digitizing frequency, the measurement frequency and the digitizing frequency being a measurement frequency and a digitizing frequency of the respective tracker whose transmission frequency has been configured with the adjustment value corresponding to no transmission. Thereby, the one or more motion sensors of the respective tracker may keep taking measurements when the transmission frequency of the respective tracker has been configured with the adjustment value corresponding to no transmission, so that the respective tracker may detect when the level of motion of the tracker is not within the predetermined motion range associated with the tracker being motionless.

In some examples, the method comprises transmitting, to the computing device, by each motion tracker of the one or more motion trackers whose transmission frequency has been configured with the adjustment value corresponding to no transmission, and which has transmitted the data indicative of at least temporal no transmission of measurements to the computing device, data indicative of transmission of measurements by the respective motion tracker when the level of motion is not within the predetermined motion range associated with being motionless.

In some examples, if the computing device does not receive measurements from the motion tracker during a predetermined period of time, the data indicative of at least temporal no transmission has not been received from the tracker and the most recent measurements received, by the computing device, from the tracker are associated with a level of motion within the predetermined motion range associated with the motion tracker being motionless, then it may be considered that the body member where the tracker is arranged is motionless or substantially motionless.

In some examples, if the computing device does not receive measurements from the motion tracker during a predetermined period of time, the data indicative of at least temporal no transmission has not been received from the tracker and the most recent measurements received from the tracker are associated with a level of motion not within the predetermined motion range associated with the motion tracker being motionless, then it may be considered that the tracker is not working properly and hence it may be appropriate to halt the motion tracking system. In such cases, the method may include providing an alert of a fault condition of the motion tracking system, e.g., to a remote monitoring device.

In some examples, the method further comprises configuring, by each of the one or more motion trackers, the at least one frequency associated with the respective motion tracker at least based on a predetermined movement to be performed by the user, thereby modifying a value of the at least one predetermined frequency of the respective motion tracker. The motion tracking system may comprise data indicative of a first plurality of adjustment values for configuring the respective at least one frequency based on both a predetermined movement to be performed by the user and a body member of the user where the respective tracker is arranged, each adjustment value of the first plurality of adjustment values being associated with one predetermined movement to be performed by the user and one body member where one of the motion trackers of the plurality of motion trackers must be arranged, and each adjustment value of the first plurality of adjustment values being configured to be applied to the respective predetermined frequency of the motion tracker. In this way, the adjustment of the one or more frequencies involves applying at least one value of the first plurality of adjustment values and at least one value of the second plurality of adjustment values to the respective predetermined frequency or the respective predetermined frequencies.

In some examples, each adjustment value of the first plurality of adjustment values is further associated with the user and a parameter indicative of a level of motion of the respective tracker as measured by the one or more motion trackers of the respective tracker in a previous performance of the predetermined movement. Thereby, each adjustment value of the first plurality of adjustment values may be associated with the user and with characteristics of a previous performance, by the user, of the predetermined movement. In this way, the one or more frequencies may be adapted to each particular user, such as a user with a particular injury or with a particular disability.

In some examples, the step of configuring at least based on the predetermined movement to be performed by the user is conducted either prior to the user starting to perform the predetermined movement, or while the user performs the predetermined movement.

In some examples, the method further comprises: taking and/or digitizing and/or processing and/or transmitting to the computing device measurements, by each of the one or more motion trackers, at the respective frequency of the at least one frequency.

A third aspect of the disclosure provides a device or system comprising: means or components adapted to execute the method according to the first or the second aspect of the disclosure.

A fourth aspect of the disclosure provides a motion tracking system comprising a plurality of motion trackers, each motion tracker of the plurality of motion trackers being adapted to be arrangeable on a body member of a user. Each motion tracker may include at least one processor, at least one memory, at least one sensor comprising one or more motion sensors configured to measure motion of the one or more motion sensors. The system may include a computing device. The computing device and the at least one processor may be configured to conduct the method of the first or second aspect of the disclosure.

In some examples, each motion tracker of the plurality of motion trackers comprises a predetermined measurement frequency with which at least one sensor of the respective motion tracker measures a respective physical magnitude, a predetermined digitizing frequency with which the respective tracker digitizes measurements, a predetermined processing frequency with which the respective tracker processes digital measurements, and/or a predetermined transmission frequency with which the respective tracker transmits measurements to the computing device of the motion tracking system.

A fifth aspect of the disclosure provides a computer program product that has instructions which, when executed by at least one motion tracking device or motion tracking system comprising at least one processor, cause the at least one motion tracking device or motion tracking system to carry out the steps of a method according to the first or second aspect of the disclosure.

Similar benefits as those described for the first and second aspects of the disclosure may also be applicable to the third and/or fourth and/or fifth aspects of the disclosure.

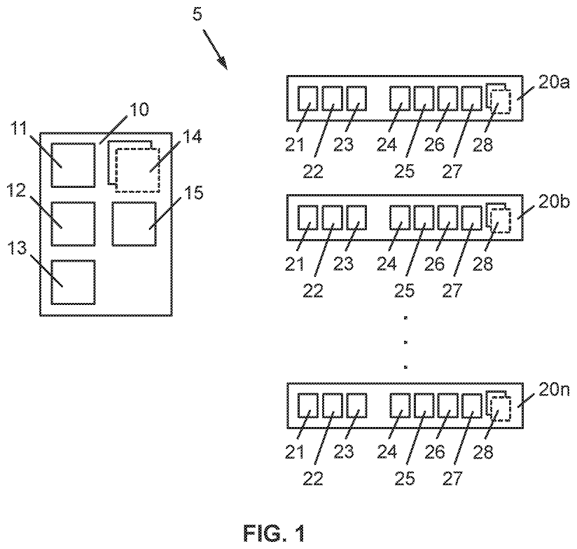

FIG. 1 diagrammatically shows a motion tracking system 5 in accordance with some examples. The motion tracking system 5 includes a computing device 10, which may, for example, be a tablet, a mobile phone, or a personal computer, and one or more trackers 20a-20n.

Each tracker 20a-20n comprises at least one sensor, for example, an inertial measurement unit. The inertial measurement unit of each tracker comprises one or more inertial sensors selected from, for example, an accelerometer 21, a gyroscope 22, and a magnetometer 23. In FIG. 1, each tracker 20a-20n comprises an inertial measurement unit comprising all three inertial sensors 21-23, but in other examples the trackers only include an accelerometer 21 and a gyroscope 22, for instance, or one or more other inertial sensors. In some examples, the inertial measurement unit of each tracker 20a-20n comprises the same inertial sensors 21-23.

The trackers 20a-20n each further include at least one processor 24, at least one memory 25, and a first wireless communications module 26 for transmitting radiofrequency signals to and receiving radiofrequency signals from the computing device 10. For example, the trackers 20a-20n transmit advertisement packages, data packets with identification data (e.g., one or more identities or keys), data packets with measurements of the inertial sensor(s) 21-23, data packets with directions computed by the trackers, or combinations thereof, and receive packets from the computing device 10 with, for example, at least one of: a predetermined measurement frequency with which at least one sensor of the motion tracker receiving the packets measures a respective physical magnitude, a predetermined digitizing frequency with which the tracker receiving the packets digitizes measurements taken by at least one sensor of the tracker, a predetermined processing frequency with which the tracker receiving the packets processes the digitized measurements, a predetermined transmission frequency with which the tracker receiving the packets transmits measurements taken by at least one sensor of the tracker to a computing device, instructions to configure a measurement frequency and/or a digitizing frequency and/or a processing frequency and/or a transmission frequency associated with the tracker receiving the packets, data indicative of a first plurality of adjustment values for configuring at least one of the measurement frequency, the digitizing frequency, the processing frequency and the transmission frequency associated with the tracker receiving the packets, data indicative of a second plurality of adjustment values for configuring the at least one of the measurement frequency, the digitizing frequency, the processing frequency and the transmission frequency associated with the tracker receiving the packets, data indicative of an adjustment value for configuring the measurement frequency and/or the digitizing frequency and/or the processing frequency and/or the transmission frequency associated with the tracker receiving the packets. At least when no wireless communications connections are established with the computing device 10, the radiofrequency signals of the trackers 20a-20n include advertisement packages for indicating their presence and that they are active. Once the wireless communications connections are established (using a technology and protocol known by a skilled person, for instance, but without limitation, Bluetooth and Bluetooth Low Energy communications, cellular network communications such as GSM, UMTS or LTE, wireless LAN communications, etc.) with the computing device 10, the radiofrequency signals of the trackers 20a-20n may include identification data and/or the measurements, based on which the motion tracking sequence will be provided by the computing device 10.

In some examples, each tracker 20a-20n comprises a plurality of antennas 28 for radiating and capturing electromagnetic waves as part of the operation of the first wireless communications module 26. In some other examples, each tracker 20a-20n comprises one antenna 28.

Each tracker 20a-20n is adapted to be arranged on the body of a person so that the measurements provided by each tracker 20a-20n can be processed by the computing device 10, thereby providing a motion tracking sequence of the person. The trackers 20a-20n may be attached to body members of the person by means of an attaching device 27 like, for instance, straps, Velcro (hook-and-loop fasteners), or other means, that the motion tracking system 5 or the tracker 20a-20n itself comprises.

Each tracker 20a-20n may be powered by one or more batteries, e.g., a rechargeable battery.

In some examples, at least one processor 24 of the trackers 20a-20n runs a sensor fusion algorithm for processing the digitized measurements of the inertial sensors 21-23 within the respective tracker. The sensor fusion algorithm is intended to enhance the raw measurements of the inertial sensors by correcting errors thereof due to drifts of the inertial sensors and, thus, outputs processed measurements that are to be transmitted to the computing device 10.

The computing device 10 may include at least one processor 11, at least one memory 12, and a second wireless communications module 13 for transmitting radiofrequency signals to the trackers 20a-20n and receiving radiofrequency signals therefrom. In some examples, the computing device 10 includes a plurality of antennas 14, whereas in some other examples, the computing device 10 includes one antenna 14. The antenna(s) 14 cooperate with the second wireless communications module 13.

The motion tracking system 5 may also include at least one device 15 (which may be part of the computing device 10 or be separate from the computing device 10) for providing user perceptible signals, such as a screen, loudspeakers, or a combination thereof, to name a few examples. That is to say, the at least one device 15 may comprise one or more of visual output means (e.g., screen, LEDs), audio output means (e.g., loudspeakers), vibrating means (e.g., a vibrator), or other means for providing user perceptible signals in the form of sounds, vibration, animated graphics, etc.

When the at least one device 15 comprises a screen or other display, the computing device 10 is capable of showing instructions and/or information to the intended user about the operation of the motion tracking system 5 and the motion tracking procedure to be conducted with the system 5, for example predetermined movements that are to be performed by an intended user of the motion tracking system 5, a list or representation of the body members that shall have a tracker arranged thereon for a given exercise or motion tracking procedure, results of the exercises performed by the user, etc. The device 15 may thus provide a user interface (UI) to present instructions and/or information to the user and/or to receive inputs from the user.

To this end, the computing device 10 stores, in the at least one memory 12, the predetermined body members where the trackers must be positioned, and also data relative to the physical exercises, e.g., predetermined movements, of intended users. Any of these data can be transmitted to and/or received from another electronic device thanks to the second wireless communications module 13. For example, a therapist is able to receive the feedback at a computing device in a remote location, such as a hospital, so as to monitor the evolution of the person. Based on the feedback received, the therapist is able to adjust the difficulty of the movement(s), the number of repetitions thereof, prescribe new movements, etc., so that the person may further exercise using the motion tracking system 5.

In some examples, one or more of the trackers 20a-20n may include a vital sign sensor. Examples of vital sign sensors include a respiration rate sensor, a body temperature sensor, a pulse rate sensor, or a combination of two or more thereof.

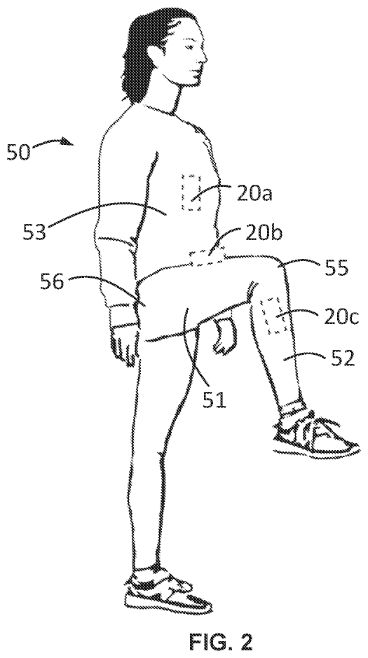

FIG. 2 shows a user (person 50) performing a first predetermined movement while wearing trackers 20a-20c of the motion tracking system 5 of FIG. 1, according to some examples.

In FIG. 2, three trackers 20a-20c are mounted, arranged or fitted on the person 50 for tracking the movement of the person 50: the first tracker 20a is on the chest 53, the second tracker 20b is on the right upper leg 51, and the third tracker 20c is on the right lower leg 52. These body members 51-53 form a kinematic chain.

In FIG. 2, a first predetermined movement to be performed by the person 50 is a knee-up, which involves the lower leg 52 and the upper leg 51 of a same leg. For example, the person 50 may select the knee-up movement in a user interface of the motion tracking system, e.g. a user interface of the computing device 10, such as the user interface provided by the device 15, thereby causing that the motion tracking system 5 generates or receives a signal which indicates that the first predetermined movement to be performed by the user is a knee-up. A user interface of the motion tracking system 5 may thus instruct the person 50 to perform a movement of one or more body members.

The motion tracking system 5 may provide a motion tracking sequence of the chest, upper leg and lower leg of the user in the performance of the first predetermined movement by the user. In this movement, the chest 53 remains still or almost still during the entire movement, and the upper leg 51 and the lower leg 52 move faster than the chest 53 and are subjected to higher accelerations and decelerations than the chest 53 in each performance of the first predetermined movement. In order to enhance accuracy of the provided motion tracking sequence while minimizing a negative impact on communications and/or power consumption, the motion tracking system 5 samples fewer data per unit of time by means of the first tracker 20a arranged on the chest 53 than by means of the second tracker 20b and the third tracker 20c arranged on the leg. In other words, a sampling frequency associated with the first tracker 20a is lower than a sampling frequency associated with the second tracker 20b and a sampling frequency associated with the third tracker 20c. The motion tracking system 5 dynamically adjusts the frequencies as needed.

If one or more sensors of the at least one sensor measure at the measurement frequency, digitizes the measurements at the digitizing frequency, processes the digitized measurements at the processing frequency and transmits the processed measurements at the transmission frequency, a sampling frequency associated with each tracker 20a-20n is dependent on the measurement frequency with which the one or more sensors of the respective motion tracker measure a respective physical magnitude, the digitizing frequency with which the respective tracker digitizes measurements taken by the at least one sensor, the processing frequency with which the respective tracker processes the digitized measurements and the transmission frequency with which the respective tracker transmits measurements taken by the at least one sensor to a computing device of the motion tracking system 5. At least one of the measurement frequency, the digitizing frequency, the processing frequency and the transmission frequency associated with each tracker 20a-20n is configured by the tracker 20a-20n by modifying a respective predetermined frequency of the tracker 20a-20n.

In some examples, the first tracker 20a has a predetermined measurement frequency with which at least one sensor of the first tracker 20a measures a respective physical magnitude, a predetermined digitizing frequency with which the first tracker 20a digitizes the measurements, a predetermined processing frequency with which the respective tracker processes the measurements and a predetermined transmission frequency with which the first tracker 20a transmits the measurements to a computing device of the motion tracking system 5. The predetermined measurement frequency of the first tracker 20a may be different from the predetermined digitizing frequency of the first tracker 20a and/or from the predetermined processing frequency of the first tracker 20a and/or from the predetermined transmission frequency of the first tracker 20a. The predetermined digitizing frequency of the first tracker 20a may be different from the predetermined processing frequency of the first tracker 20a and/or from the predetermined transmission frequency of the first tracker 20a. The predetermined processing frequency of the first tracker 20a may be different from the predetermined transmission frequency of the first tracker 20a. For the purpose of simplicity of this description, it is considered that the predetermined measurement frequency of the first tracker 20a is equal to the predetermined digitizing frequency of the first tracker 20a, equal to the predetermined processing frequency of the first tracker 20a and equal to the predetermined transmission frequency of the first tracker 20a:

predetermined measurement frequency=original_sampling_frequency

predetermined digitizing frequency=original_sampling_frequency

predetermined processing frequency=original_sampling_frequency

predetermined transmission frequency=original_sampling_frequency

The original_sampling_frequency is, for example, a default frequency associated with the first tracker 20a. For example, the original_sampling_frequency may be between 5 and 250 Hz, for example one of 10 Hz, 15 Hz, 25 Hz, 30 Hz, 40 Hz, 45 Hz, 55 Hz, 60 Hz, 70 Hz, 75 Hz, 85 Hz, 90 Hz, 100 Hz, 105 Hz, 110 Hz, 115 Hz, 120 Hz, 125 Hz, 130 Hz, 135 Hz, 140 Hz, 145 Hz, 150 Hz, 155 Hz, 160 Hz, 165 Hz, 170 Hz, 175 Hz, 180 Hz, 185 Hz, 190 Hz, 195 Hz, 200 Hz, 205 Hz, 210 Hz, 215 Hz, 220 Hz, 225 Hz, 230 Hz, 235 Hz, 240 Hz, 245 Hz or 250 Hz. In the following example it is considered that the original_sampling_frequency is 50 Hz.

The measurement frequency, the digitizing frequency, the processing frequency and the transmission frequency associated with the first tracker 20a are configured by the first tracker 20a based on one or more of:
the predetermined movement to be performed by the user 50 and a body member where the first tracker 20a is arranged or must be arranged (e.g., the chest 53), and
measurements of the motion measurement of the first tracker 20a.

The measurement frequency associated with the first tracker 20a is configured by the first tracker 20a resulting in an adjustment of the measurement frequency associated with the first tracker. The adjustment of the measurement frequency comprises applying an adjustment value a_{yyy} to the predetermined measurement frequency. The adjustment value a_{yyy} is configured to be applied to the predetermined measurement frequency of the first tracker 20a.

In some examples, the adjustment value is a first adjustment value a₁₁₁ of a first plurality of adjustment values a₁₁₁, a₁₁₂, . . . a₁₁ₘ, a₁₂₁, a₁₂₂, . . . a₁ₚₘ; each adjustment value of the first plurality of adjustment values being associated with one predetermined movement to be performed by the user and one body member where one of the first tracker 20a, the second tracker 20b and the third tracker 20c is arranged or must be arranged. At least some of the first plurality of adjustment values are configured to be applied to the predetermined measurement frequency of the first tracker 20a and are associated with the chest (e.g., the body member where the first tracker 20 is arranged). In FIG. 2, the first adjustment value a₁₁₁ is associated with the knee-up movement and with the chest (e.g., the body member where the first tracker 20a is arranged).

In some examples, if the predetermined measurement frequency is appropriate for sampling a chest motion in a movement in which the chest moves relatively faster (e.g., a push-up) when compared to a knee-up, then the application of the first adjustment value a₁₁₁ to the predetermined measurement frequency results in a measurement frequency which is lower than the predetermined measurement frequency. In some examples, each adjustment value of the first plurality of adjustment values is a factor configured to multiply the respective predetermined frequency, the first adjustment value a₁₁₁ being, for example, 0.5:

measurement frequency=predetermined measurement frequency·a₁₁₁=50·0.5=25 (Hz)

This results in a measurement frequency of the first tracker 20a of 25 (Hz).

In some examples, the adjustment value of the first plurality of adjustment values configured to be applied to the predetermined digitizing frequency of the first tracker 20a and associated with the knee-up movement and with the chest is the first adjustment value a₁₁₁:

digitizing frequency=predetermined digitizing frequency·a₁₁₁

In some examples, the adjustment value of the first plurality of adjustment values configured to be applied to the predetermined digitizing frequency of the first tracker 20a and associated with the knee-up movement and with the chest is lower or higher than the first adjustment value a₁₁₁.

In some examples, the adjustment value of the first plurality of adjustment values configured to be applied to the predetermined processing frequency of the first tracker 20a and associated with the knee-up movement and with the chest is the first adjustment value a₁₁₁:

processing frequency=predetermined processing frequency·a₁₁₁

In some examples, the adjustment value of the first plurality of adjustment values configured to be applied to the predetermined processing frequency of the first tracker 20a and associated with the knee-up movement and with the chest is lower or higher than the first adjustment value a₁₁₁.

In some examples, the adjustment value of the first plurality of adjustment values configured to be applied to the predetermined transmission frequency of the first tracker 20a and associated with the knee-up movement and with the chest is the first adjustment value a₁₁₁:

transmission frequency=predetermined transmission frequency·a₁₁₁

In some examples, the adjustment value of the first plurality of adjustment values configured to be applied to the predetermined transmission frequency of the first tracker 20a and associated with the knee-up movement and with the chest is lower or higher than the first adjustment value a₁₁₁.

The predetermined measurement frequency associated with the second tracker 20b may have a different value than the predetermined measurement frequency associated with the first tracker 20a. For the purpose of simplicity of this description, it is considered that the predetermined measurement frequency of the first tracker 20a is equal to the predetermined measurement frequency of the second tracker 20b. Since in the knee-up movement the upper leg is subjected to a higher level of motion, e.g., a higher speed, than the chest, a second adjustment value a₁₁₂ of the first plurality of adjustment values configured to be applied to the predetermined measurement frequency of the second tracker 20b and associated with the knee-up movement and with the upper leg is higher than the first adjustment value; the second adjustment value is, for example, 1.5:

measurement frequency=predetermined measurement frequency·a₁₁₂=50·1.5=75 (Hz)

In some examples, the adjustment value of the first plurality of adjustment values configured to be applied to the predetermined digitizing frequency of the second tracker 20b and associated with the knee-up movement and with the upper leg is the second adjustment value a₁₁₂:

digitizing frequency=predetermined digitizing frequency·a₁₁₂

In some examples, the adjustment value of the first plurality of adjustment values configured to be applied to the predetermined digitizing frequency of the second tracker 20b and associated with the knee-up movement and with the upper leg is lower or higher than the second adjustment value a₁₁₂.

In some examples, the adjustment value of the first plurality of adjustment values configured to be applied to the predetermined processing frequency of the second tracker 20b and associated with the knee-up movement and with the upper leg is the second adjustment value a₁₁₂:

processing frequency=predetermined processing frequency·a₁₁₂

In some examples, the adjustment value of the first plurality of adjustment values configured to be applied to the predetermined processing frequency of the second tracker 20b and associated with the knee-up movement and with the upper leg is lower or higher than the second adjustment value a₁₁₂.

In some examples, the adjustment value of the first plurality of adjustment values configured to be applied to the predetermined transmission frequency of the second tracker 20b and associated with the knee-up movement and with the upper leg is the second adjustment value a₁₁₂:

transmission frequency=predetermined transmission frequency·a₁₁₂

In some examples, the adjustment value of the first plurality of adjustment values configured to be applied to the predetermined transmission frequency of the second tracker 20b and associated with the knee-up movement and with the upper leg is lower or higher than the second adjustment value a₁₁₂.

The measurement frequency associated with the second tracker 20b and resulting from the adjustment associated with the second tracker 20b is higher than the measurement frequency associated with the first tracker 20a and resulting from the adjustment associated with the first tracker 20a, the digitizing frequency associated with the second tracker 20b and resulting from the adjustment associated with the second tracker 20b is higher than the digitizing frequency associated with the first tracker 20a and resulting from the adjustment associated with the first tracker 20a, the processing frequency associated with the second tracker 20b and resulting from the adjustment associated with the second tracker 20b is higher than the processing frequency associated with the first tracker 20a and resulting from the adjustment associated with the first tracker 20a, and the transmission frequency associated with the second tracker 20b and resulting from the adjustment associated with the second tracker 20b is higher than the transmission frequency associated with the first tracker 20a and resulting from the adjustment associated with the first tracker 20a. In the case of FIG. 2, some reasons for these differences are that:
the upper leg 51 is subjected to higher accelerations than the chest 53, and/or
the upper leg 51 is subjected to higher decelerations than the chest 53, and/or
the upper leg 51 moves faster than the chest 53.

In some examples, such as cases in which the predetermined measurement frequency associated with the first tracker 20a is equal to the predetermined measurement frequency associated with the second tracker 20b, the predetermined digitizing frequency associated with the first tracker 20a is equal to the predetermined digitizing frequency associated with the second tracker 20b, the predetermined processing frequency associated with the first tracker 20a is equal to the predetermined processing frequency associated with the second tracker 20b and the predetermined transmission frequency associated with the first tracker 20a is equal to the predetermined transmission frequency associated with the second tracker 20b, the relatively higher frequencies associated with the second tracker 20b may be obtained by applying an adjustment value of the first plurality of adjustment values to each of the predetermined frequencies, each adjustment value being higher than the adjustment value of the first plurality of adjustment values applied to the respective frequency of the first tracker 20a.

A third adjustment value a₁₁₃ of the first plurality of adjustment values configured to be applied to the predetermined measurement frequency of the third tracker 20c and associated with the knee-up movement and with the lower leg is higher than the second adjustment value; the third adjustment value is, for example, 2:

measurement frequency=predetermined measurement frequency·a₁₁₃=50·2=100 (Hz)

For example, the third adjustment value a₁₁₃ is higher than the second adjustment value a₁₁₂ because the knee-up movement subjects the lower leg 52 to higher accelerations, decelerations and speed compared to the accelerations, decelerations and speed of the upper leg 51.

In some examples, the adjustment value of the first plurality of adjustment values configured to be applied to the predetermined digitizing frequency of the third tracker 20c and associated with the knee-up movement and with the lower leg is the third adjustment value a₁₁₃:

digitizing frequency=predetermined digitizing frequency·a₁₁₃

In some examples, the adjustment value of the first plurality of adjustment values configured to be applied to the predetermined digitizing frequency of the third tracker 20c and associated with the knee-up movement and with the lower leg is lower or higher than the third adjustment value a₁₁₃.

In some examples, the adjustment value of the first plurality of adjustment values configured to be applied to the predetermined processing frequency of the third tracker 20c and associated with the knee-up movement and with the lower leg is the third adjustment value a₁₁₃:

processing frequency=predetermined processing frequency·a₁₁₃

In some examples, the adjustment value of the first plurality of adjustment values configured to be applied to the predetermined processing frequency of the third tracker 20c and associated with the knee-up movement and with the lower leg is lower or higher than the third adjustment value a₁₁₃.

In some examples, the adjustment value of the first plurality of adjustment values configured to be applied to the predetermined transmission frequency of the third tracker 20c and associated with the knee-up movement and with the lower leg is the third adjustment value a₁₁₃:

transmission frequency=predetermined transmission frequency·a₁₁₃

In some examples, the adjustment value of the first plurality of adjustment values configured to be applied to the predetermined transmission frequency of the third tracker 20c and associated with the knee-up movement and with the lower leg is lower or higher than the third adjustment value a₁₁₃.

The measurement frequency associated with the third tracker 20c and resulting from the adjustment associated with the third tracker 20c is higher than the measurement frequency associated with the second tracker 20b and resulting from the adjustment associated with the second tracker 20b, the digitizing frequency associated with the third tracker 20c and resulting from the adjustment associated with the third tracker 20c is higher than the digitizing frequency associated with the second tracker 20b and resulting from the adjustment associated with the second tracker 20b, the processing frequency associated with the third tracker 20c and resulting from the adjustment associated with the third tracker 20c is higher than the processing frequency associated with the second tracker 20b and resulting from the adjustment associated with the second tracker 20b, and the transmission frequency associated with the third tracker 20c and resulting from the adjustment associated with the third tracker 20c is higher than the transmission frequency associated with the second tracker 20b and resulting from the adjustment associated with the second tracker 20b. In the case of FIG. 2, some reasons for these differences are that:
the lower leg 52 is subjected to higher accelerations than the upper leg 51, and/or
the lower leg 52 is subjected to higher decelerations than the upper leg 51, and/or
the lower leg 52 moves faster than the upper leg 51.

Although in the previous examples it has been considered that the trackers arranged at body members which move with higher level of motion (e.g., at least one of: faster, with higher linear acceleration, with higher linear deceleration, with higher variation of linear acceleration, with higher variation of linear deceleration, with higher angular acceleration, with higher angular deceleration, with higher variation of angular acceleration and with higher variation of angular deceleration) in the predetermined movement are associated with a higher frequency resulting from the adjustment associated with the particular tracker, in other examples the frequency resulting from the adjustment of the frequency and associated with trackers arranged in body members which move with higher level of motion is lower than the frequency resulting from the adjustment of frequency associated with trackers arranged in body members which move with lower intensity. For example, it may be appropriate to sample movement of a neck at a higher frequency than other more robust body members which move with higher intensity because the neck is relatively weaker and slightly wrong movements of the neck may have a relatively high impact on e.g. rehabilitation movements.

In some examples, such as cases in which the predetermined measurement frequency associated with the second tracker 20b is equal to the predetermined measurement frequency associated with the third tracker 20c, the predetermined digitizing frequency associated with the second tracker 20b is equal to the predetermined digitizing frequency associated with the third tracker 20c, the predetermined processing frequency associated with the second tracker 20b is equal to the predetermined processing frequency associated with the third tracker 20c and the predetermined transmission frequency associated with the second tracker 20b is equal to the predetermined transmission frequency associated with the third tracker 20c, the relatively higher frequencies associated with the third tracker 20c may be obtained by applying an adjustment value to each of the predetermined frequencies, each adjustment value being higher than the adjustment value applied to the respective predetermined frequency of the second tracker 20b.

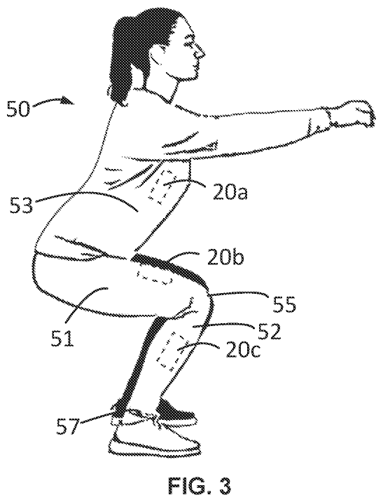

FIG. 3 shows the person 50 performing a second predetermined movement while wearing trackers 20a-20c of the motion tracking system 5, according to some examples.

In FIG. 3, the person 50 has the three trackers 20a-20c arranged as in FIG. 2, the difference being that the second predetermined movement is a squat, which involves the lower legs or shanks 52, and the upper legs or thighs 51, and the chest 53. In this movement, the end 57 of the lower legs 52 that connects to the ankle has a known position that remains still or almost still during the movement.

In a squat, the chest generally moves faster and is subjected to higher accelerations and decelerations than in the knee-up. The adjustment value of the first plurality of adjustment values associated with the squat and with the chest is higher than the first adjustment value a₁₁₁, for example, is equal to 1.

The speed, acceleration and deceleration of the upper leg in the squat may have similar modulus than in the knee-up. The adjustment value of the first plurality of adjustment values associated with the squat and with the upper leg is similar to the second adjustment value a₁₁₂, for example, is equal to 1.5.

In a squat, the lower leg moves slower and is subjected to lower accelerations and decelerations than in the knee-up. The adjustment value of the first plurality of adjustment values associated with the squat and with the lower leg is lower than the third adjustment value a₁₁₃, for example, is equal to 0.5.

The adjustment value of the first plurality of adjustment values is determined based on data indicative of the first plurality of adjustment values, the predetermined movement to be performed by the user, and the body member of the user where the respective tracker is arranged or must be arranged.

The data indicative of the first plurality of adjustment values may be stored in a memory, for example, in a memory 25 of the at least one tracker 20a-20c of the motion tracking system and/or in a memory of a computing device e.g. the memory 12 of the computing device 10. In some examples, the at least one tracker 20a-20c of the motion tracking system 5 receives the data indicative of the first plurality of adjustment values from the computing device, e.g., the computing device 10. The data indicative of the first plurality of adjustment values may comprise the first plurality of adjustment values and relationships of each adjustment value of the first plurality of adjustment values with one predetermined movement and with one body member.

In some examples, the tracker determines an adjustment value based on the first plurality of adjustment values stored in the memory 25 of the tracker and based on a predetermined movement to be performed by the user and a body member of the user where the tracker is arranged or must be arranged. For example, the first tracker 20a may determine the first adjustment value based on the knee-up movement and the chest.

In some examples, the motion tracking is performed by using measurements received from the motion trackers, data indicative of lengths of body members and data indicative of position and orientation of reference points of the body; the measurements being measurements of at least one of: acceleration obtained via the accelerometers of the trackers 20a, 20b, 20c, measurements of angular velocity obtained via the gyroscopes of the trackers 20a, 20b, 20c and measurements of orientation obtained via the gyroscopes of the trackers 20a, 20b, 20c.

For example, a position of the lower leg of a user may be estimated in the following manner in the performance of a knee-up. The computing device 10 receives predetermined data indicative of a length of the upper leg 51 and data indicative of a length of the lower leg 52. The computing device 10 receives data indicative of an initial position of the end 56 of the upper leg 51 that connects to the hip (see FIG. 2). Since in the knee-up movement the position of the end 56 remains still or substantially still, the position of the end 56 may be approximated to a constant position along the knee-up movement. The computing device 10 may receive, from the second tracker 20b, data indicative of an orientation of the second tracker 20b and hence of the upper leg 51. The computing device 10 estimates the position of the knee 55 of the user from the data indicative of the orientation of the upper leg 51, the data indicative of the length of the upper leg 51 and the position of the end 56 of the upper leg 51 that connects to the hip. The computing device 10 receives data indicative of the orientation of the third tracker 20c and hence of the orientation of the lower leg 52. The computing device 10 estimates a position of the lower leg 52 based on the estimated position of the knee 55, on the data indicative of the orientation of the lower leg 52 and on the data indicative of a length of the lower leg 52.

In some examples, the computing device, e.g. the computing device 10, obtains data indicative of an adjustment value of the first plurality of adjustment values, the adjustment value being associated with a predetermined movement to be performed by the user and a body member of the user where the respective motion tracker is arranged or must be arranged. The tracker arranged in the body member receives the data indicative of the adjustment value. For example, the first tracker 20a may determine the first adjustment value based on received data indicative of the first adjustment value. In some examples, the data indicative of the first adjustment value is the first adjustment value.

The determination of an adjustment value of the first adjustment values may comprise retrieving the adjustment value associated with the predetermined movement to be performed by the user and the body member where the respective motion tracker is arranged or must be arranged. The retrieving is performed, for example, by means of executing an instruction for retrieving data stored in a memory, the instruction comprising data indicative of the predetermined movement to be performed by the user and the body member where the respective motion tracker is arranged or must be arranged. The instruction is, for example, a query for retrieving data from a database stored in a memory.

In some examples, each adjustment value of the first plurality of adjustment values is further associated with the user and a parameter indicative of a level of motion of the respective tracker as measured by the inertial measurement unit of the respective tracker in a previous performance of the predetermined movement. For example, prior to determining and/or updating the first adjustment value, the user may perform one or more repetitions of a knee-up. The first tracker 20a obtains inertial measurements of the movement of the chest 53 in the repetitions. One or more levels of motion of the chest are obtained based on the inertial measurements. If the user has, for example, an injury in the back, the movement of the chest 53 in the one or more repetitions may be slower compared to the same movement performed by the same user without the back injury. Accordingly, accuracy of the motion tracking system may barely decrease by decreasing the measurement frequency, the digitizing frequency, the processing frequency and/or the transmission frequency associated with the first tracker 20a arranged in the chest 53 of the injured user compared with an accuracy obtained with the respective measurement frequency, digitizing frequency, processing frequency and/or transmission frequency associated with the first tracker 20a arranged in the chest of the same user without the injury.

In some examples, the measurement frequency, the digitizing frequency, the processing frequency and/or the transmission frequency of a motion tracker of the one or more motion trackers is configured based on measurements of the inertial measurement of the motion tracker. In particular, the frequency of the tracker is configured by applying an adjustment value of the second plurality of adjustment values to the respective predetermined frequency of the tracker. Each adjustment value of the second plurality of adjustment values is associated with a level of motion of the tracker as measured by the inertial measurement unit of the tracker, the level of motion of the motion tracker being within a predetermined motion range. An example is explained below.

In a knee-up movement, the chest 53 moves slower than the lower leg 52 and is subjected to lower accelerations and decelerations than the lower leg 52. The motion tracking system 5 may estimate a level of motion, e.g., speed and/or linear accelerations and/or angular accelerations, of the chest 53 based on measurements taken by the inertial measurement unit of the first tracker 20a. The motion tracking system 5 may estimate a level of motion of the lower leg 52 based on measurements taken by the inertial measurement unit of the third tracker 20c. Considering that the predetermined measurement frequency of the first tracker 20a is equal to the predetermined measurement frequency of the third tracker 20c, the fourth adjustment value a₂₁₁ of the second plurality of adjustment values associated with the level of motion of the first tracker 20a is lower than a fifth adjustment value a₂₂₁ of the second plurality of adjustment values associated with the third tracker 20c. For example, the fourth adjustment value a₂₁₁ may be 0.5 and the fifth adjustment value a₂₂₁ may be 1.5:

measurement frequency of first tracker=predetermined measurement frequency·a₂₁₁=50·0.5=25 Hz

measurement frequency of third tracker=predetermined measurement frequency·a₂₂₁=50·1.5=75 Hz

In some examples, the user performs the predetermined movement slower or faster than it is expected according to the first plurality of adjustment values associated with the predetermined movement and the body member where the tracker is arranged. For example, the user performs the knee-up movement raising the leg slowly. Since the first plurality of adjustment values do not take into account this situation, an adjustment of the measurement frequency, digitizing frequency, processing frequency and/or transmission frequency of the third tracker 20c by applying the corresponding adjustment value of the first plurality of adjustment values will result in a frequency higher than required. To overcome this limitation of the first plurality of adjustment values, an adjustment value of the second plurality of adjustment values may be applied to the respective predetermined frequency. In this case, both the third adjustment value a₁₁₃ of the first plurality of adjustment values and a sixth adjustment value a₂₃₁ of the second plurality of adjustment values are applied to the respective predetermined frequency, e.g. the predetermined measurement frequency.

measurement frequency of first tracker=predetermined measurement freq·a₁₁₃·a₂₃₁=50·2·0.5=50 Hz

Accordingly, in some examples, as the person 50 carries out relevant exercise/s (e.g., based on instructions transmitted via the user interface), additional adjustment of one or more frequencies of one or more of the plurality of motion trackers may be performed. For example, additional adjustments may be made by the motion tracking system 5 to further reduce the power consumption of at least one of the motion trackers.

The additional adjustment may be based on a measured level of motion of the one or more motion trackers. A magnitude of the additional adjustment may be based on the measured level of motion and/or a range of motion associated with the movement.

The motion tracking system 5 may thus dynamically and selectively adjust frequencies. Frequencies may be repeatedly adjusted during operation of the motion tracking system 5, e.g., to maintain at least a threshold motion tracking accuracy level while increasing a life of a battery of one or more of the trackers 20a-20n.

If the level of motion of the first tracker 20a decreases a lot, for example, because the user remains still, then the level of motion of the first tracker 20a is within a predetermined motion range associated with the chest 53 being motionless. In this case, waste of power consumed by the first tracker 20a may be minimized by applying a seventh adjustment value a₂₁₂ of the second plurality of adjustment values to the predetermined transmission frequency of the first tracker 20a, the seventh adjustment value a₂₁₂ corresponding to no transmission. For example, the seventh adjustment value a₂₁₂ may be zero:

transmission frequency=predetermined transmission frequency·a₂₁₂=50·0=0 Hz

Upon setting the transmission frequency to zero, the first tracker 20a may transmit, to the computing device, data indicative of at least temporal no transmission of measurements.

Although specific examples are described herein, it will be evident that various modifications and changes may be made to these examples without departing from the broader spirit and scope of the disclosure. Accordingly, the specification and drawings are to be regarded in an illustrative rather than a restrictive sense. The accompanying drawings that form a part hereof show by way of illustration, and not of limitation, specific examples in which the subject matter may be practiced. The examples illustrated are described in sufficient detail to enable those skilled in the art to practice the teachings disclosed herein. Other examples may be utilized and derived therefrom, such that structural and logical substitutions and changes may be made without departing from the scope of this disclosure. This detailed description, therefore, is not to be taken in a limiting sense, and the scope of various examples is defined only by the appended claims, along with the full range of equivalents to which such claims are entitled.

Such examples of the inventive subject matter may be referred to herein, individually or collectively, by the term “example” merely for convenience and without intending to voluntarily limit the scope of this application to any single example or concept if more than one is in fact disclosed. Thus, although specific examples have been illustrated and described herein, it should be appreciated that any arrangement calculated to achieve the same purpose may be substituted for the specific examples shown. This disclosure is intended to cover any and all adaptations or variations of various examples. Combinations of the above examples, and other examples not specifically described herein, will be apparent to those of skill in the art upon reviewing the above description.

Some portions of the subject matter discussed herein may be presented in terms of algorithms or symbolic representations of operations on data stored as bits or binary digital signals within a machine memory (e.g., a computer memory). Such algorithms or symbolic representations are examples of techniques used by those of ordinary skill in the data processing arts to convey the substance of their work to others skilled in the art. As used herein, an “algorithm” is a self-consistent sequence of operations or similar processing leading to a desired result. In this context, algorithms and operations involve physical manipulation of physical quantities. Typically, but not necessarily, such quantities may take the form of electrical, magnetic, or optical signals capable of being stored, accessed, transferred, combined, compared, or otherwise manipulated by a machine. It is convenient at times, principally for reasons of common usage, to refer to such signals using words such as “data,” “content,” “bits,” “values,” “elements,” “symbols,” “characters,” “terms,” “numbers,” “numerals,” or the like. These words, however, are merely convenient labels and are to be associated with appropriate physical quantities.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or any suitable combination thereof), registers, or other machine components that receive, store, transmit, or display information. Furthermore, unless specifically stated otherwise, the terms “a” and “an” are herein used, as is common in patent documents, to include one or more than one instance. As used herein, the conjunction “or” refers to a non-exclusive “or,” unless specifically stated otherwise.

Unless the context clearly requires otherwise, throughout the description and the claims, the words “comprise,” “comprising,” and the like are to be construed in an inclusive sense, as opposed to an exclusive or exhaustive sense, e.g., in the sense of “including, but not limited to.” As used herein, the terms “connected,” “coupled,” or any variant thereof means any connection or coupling, either direct or indirect, between two or more elements; the coupling or connection between the elements can be physical, logical, or a combination thereof. Additionally, the words “herein,” “above,” “below,” and words of similar import, when used in this application, refer to this application as a whole and not to any particular portions of this application. Where the context permits, words using the singular or plural number may also include the plural or singular number, respectively. The word “or” in reference to a list of two or more items, covers all of the following interpretations of the word: any one of the items in the list, all of the items in the list, and any combination of the items in the list.

Although some examples may include a particular sequence of operations, the sequence may be altered without departing from the scope of the present disclosure. For example, some of the operations depicted may be performed in parallel or in a different sequence that does not materially affect the functions as described in the examples. In other examples, different components of an example device or system that implements an example method may perform functions at substantially the same time or in a specific sequence.

As used herein, the term “processor” may refer to any one or more circuits or virtual circuits (e.g., a physical circuit emulated by logic executing on an actual processor) that manipulates data values according to control signals (e.g., commands, opcodes, machine code, control words, macroinstructions, etc.) and which produces corresponding output signals that are applied to operate a machine. A processor may, for example, include at least one of a Central Processing Unit (CPU), a Reduced Instruction Set Computing (RISC) Processor, a Complex Instruction Set Computing (CISC) Processor, a Graphics Processing Unit (GPU), a Digital Signal Processor (DSP), a Tensor Processing Unit (TPU), a Neural Processing Unit (NPU), a Vision Processing Unit (VPU), a Machine Learning Accelerator, an Artificial Intelligence Accelerator, an Application Specific Integrated Circuit (ASIC), a Field Programmable Gate Array (FPGA), a Radio-Frequency Integrated Circuit (RFIC), a Neuromorphic Processor, a Quantum Processor, or any combination thereof. A processor may be a multi-core processor having two or more independent processors (sometimes referred to as “cores”) that may execute instructions contemporaneously. Multi-core processors may contain multiple computational cores on a single integrated circuit die, each of which can independently execute program instructions in parallel. Parallel processing on multi-core processors may be implemented via architectures like superscalar, VLIW, vector processing, or SIMD that allow each core to run separate instruction streams concurrently. A processor may be emulated in software, running on a physical processor, as a virtual processor or virtual circuit. The virtual processor may behave like an independent processor but is implemented in software rather than hardware.

The various operations of example methods described herein may be performed, at least partially, by one or more processors that are temporarily configured (e.g., by software) or permanently configured to perform the relevant operations. Whether temporarily or permanently configured, such processors may constitute processor-implemented modules/components that operate to perform one or more operations or functions. The modules/components referred to herein may, in some examples, comprise processor-implemented modules/components.

Similarly, the methods described herein may be at least partially processor-implemented. For example, at least some of the operations of a method may be performed by one or more processors or processor-implemented modules/components. The performance of certain of the operations may be distributed among the one or more processors, not only residing within a single machine, but deployed across a number of machines. In some examples, the processor or processors may be located in a single location (e.g., within a home environment, an office environment, or a server farm), while in other examples the processors may be distributed across a number of locations.

Examples may be implemented in digital electronic circuitry, or in computer hardware, firmware, or software, or in combinations of them. Examples may be implemented using a computer program product, e.g., a computer program tangibly embodied in an information carrier, e.g., in a machine-readable medium for execution by, or to control the operation of, data processing apparatus, e.g., a programmable processor, a computer, or multiple computers.

A computer program can be written in any form of programming language, including compiled or interpreted languages, and it can be deployed in any form, including as a standalone program or as a module, subroutine, or other unit suitable for use in a computing environment. A computer program can be deployed to be executed on one computer or on multiple computers at one site or distributed across multiple sites and interconnected by a communication network.

When an ordinal number (such as “first”, “second”, “third” and so on) is used as an adjective before a term or expression, that ordinal number is used (unless expressly specified otherwise) merely to indicate a particular feature, such as to distinguish that particular feature from another feature that is described by the same term or expression or by a similar term or expression. For example, a “first widget” may be so named merely to distinguish it from, e.g., a “second widget”. Thus, the mere usage of the ordinal numbers “first” and “second” before the term “widget” does not indicate any other relationship between the two widgets, and likewise does not indicate any other characteristics of either or both widgets. For example, the mere usage of the ordinal numbers “first” and “second” before the term “widget” does not indicate that either widget comes sequentially before or after any other in order or location; does not indicate that either widget occurs or acts before or after any other in time; and does not indicate that either widget ranks above or below any other, as in importance or quality. In addition, the mere usage of ordinal numbers does not define a numerical limit to the features identified with the ordinal numbers. For example, the mere usage of the ordinal numbers “first” and “second” before the term “widget” does not indicate that there must be no more than two widgets and the mere usage of the ordinal number “second” before the term “widget” does not indicate that there must be a “first widget”.

In view of the above-described implementations of subject matter this application discloses the following list of examples, wherein one feature of an example in isolation or more than one feature of an example, taken in combination and, optionally, in combination with one or more features of one or more further examples are further examples also falling within the disclosure of this application.

Example 1 is a method for reducing a power consumption of a motion tracking system, the motion tracking system comprising a computing device and a plurality of motion trackers in communication with the computing device, and the method comprising: using a user interface provided by the computing device to instruct a subject to perform a movement of one or more body members of the subject; performing an adjustment of one or more frequencies of one or more of the plurality of motion trackers to reduce a power consumption of at least one of the plurality of motion tracker to thereby reduce the power consumption of the motion tracking system, wherein the one or more frequencies are adjusted based at least in part on the movement which the subject is instructed to perform; and during or subsequent to performing the adjustment of the one or more frequencies, tracking the subject using the plurality of motion trackers of the motion tracking system, wherein, during the tracking of the subject, one or more of the plurality of motion trackers is disposed on or adjacent to the one or more body members of the subject.

In Example 2, the subject matter of Example 1 includes, wherein the one or more frequencies are adjusted by one or more corresponding adjustment values based at least in part on a body member of the one or more body members associated with the one or more motion trackers.

In Example 3, the subject matter of any of Examples 1-2 includes, wherein the adjustment of the one or more frequencies comprises adjusting the one or more frequencies of each of the plurality of motion trackers.

In Example 4, the subject matter of any of Examples 1-3 includes, wherein the adjustment of the one or more frequencies comprises adjusting the one or more frequencies based at least in part on a level of motion associated with the subject, as determined by a previously recorded measurement of the subject by the motion tracking system.

In Example 5, the subject matter of any of Examples 1-4 includes, wherein the tracking of the subject is performed during the adjustment of the one or more frequencies.

In Example 6, the subject matter of Example 5 includes, subsequent to the adjustment of the one or more frequencies, performing an additional adjustment of the one or more frequencies of one or more of the plurality of motion trackers during the performance of the movement by the subject to further reduce the power consumption of the at least one of the plurality of motion trackers.

In Example 7, the subject matter of Example 6 includes, wherein the additional adjustment is based at least in part on a measured level of motion of the one or more motion trackers.

In Example 8, the subject matter of Example 7 includes, wherein a magnitude of the additional adjustment is based on the measured level of motion and a range of motion associated with the movement which the subject is instructed to perform.

In Example 9, the subject matter of any of Examples 1-8 includes, adjusting a transmission frequency of at least one of the plurality of motion trackers such that substantially no transmission occurs when the at least one of the plurality of motion trackers is motionless.

In Example 10, the subject matter of Example 9 includes, detecting, by the motion tracking system, that the at least one of the plurality of motion trackers is motionless; determining that the at least one of the plurality of motion trackers is not expected to be motionless within a tracked motion range associated with the movement which the subject is instructed to perform; halting tracking of the movement; and providing an alert of a fault condition of the motion tracking system.

In Example 11, the subject matter of any of Examples 1-10 includes, wherein the plurality of motion trackers each comprises an inertial measurement unit.

In Example 12, the subject matter of Example 11 includes, wherein the inertial measurement unit comprises at least two of an accelerometer, a gyroscope, and a magnetometer.

In Example 13, the subject matter of any of Examples 1-12 includes, wherein at least one of the plurality of motion trackers comprises a vital sign sensor.

In Example 14, the subject matter of Example 13 includes, wherein the vital sign sensor comprises a respiration rate sensor, a body temperature sensor, a pulse rate sensor, or a combination of two or more thereof.

In Example 15, the subject matter of Example 14 includes, wherein the one or more frequencies are repeatedly adjusted during operation of the motion tracking system.

In Example 16, the subject matter of Example 15 includes, wherein the one or more frequencies are repeatedly adjusted to maintain at least a threshold motion tracking accuracy level while increasing a life of a battery of each of the plurality of motion trackers.

In Example 17, the subject matter of Example 16 includes, wherein a hardware configuration of each of the plurality of motion trackers is identical.

In Example 18, the subject matter of any of Examples 1-17 includes, wherein each of the plurality of motion trackers is powered by a battery.

Example 19 is a motion tracking system comprising at least one computing device and a plurality of motion trackers in communication with the at least one computing device, and configured to perform operations comprising: using a user interface provided by the computing device to instruct a subject to perform a movement of one or more body members of the subject; performing an adjustment of one or more frequencies of one or more of the plurality of motion trackers to reduce a power consumption of at least one of the plurality of motion tracker to thereby reduce a power consumption of the motion tracking system, wherein the one or more frequencies are adjusted based at least in part on the movement which the subject is instructed to perform; and during or subsequent to performing the adjustment of the one or more frequencies, tracking the subject using the plurality of motion trackers of the motion tracking system, wherein, during the tracking of the subject, one or more of the plurality of motion trackers is disposed on or adjacent to the one or more body members of the subject.

Example 20 is a non-transitory computer readable medium comprising instructions that, when executed by at least one computer processor, cause the at least one computer processor to perform operations comprising: in a motion tracking system comprising a computing device and a plurality of motion trackers in communication with the computing device, using a user interface provided by the computing device to instruct a subject to perform a movement of one or more body members of the subject; performing an adjustment of one or more frequencies of one or more of the plurality of motion trackers to reduce a power consumption of at least one of the plurality of motion tracker to thereby reduce a power consumption of the motion tracking system, wherein the one or more frequencies are adjusted based at least in part on the movement which the subject is instructed to perform; and during or subsequent to performing the adjustment of the one or more frequencies, tracking the subject using the plurality of motion trackers of the motion tracking system, wherein, during the tracking of the subject, one or more of the plurality of motion trackers is disposed on or adjacent to the one or more body members of the subject.

Example 21 is at least one machine-readable medium including instructions that, when executed by processing circuitry, cause the processing circuitry to perform operations to implement any of Examples 1-20.

Example 22 is an apparatus comprising means to implement any of Examples 1-20.

Example 23 is a system to implement any of Examples 1-20.

Example 24 is a method to implement any of Examples 1-20.

1. A method for reducing a power consumption of a motion tracking system, the motion tracking system comprising a computing device and a plurality of motion trackers in communication with the computing device, and the method comprising:
using a user interface provided by the computing device to instruct a subject to perform a movement of one or more body members of the subject;
performing an adjustment of one or more frequencies of one or more of the plurality of motion trackers to reduce a power consumption of at least one of the plurality of motion tracker to thereby reduce the power consumption of the motion tracking system, wherein the one or more frequencies is adjusted based at least in part on the movement which the subject is instructed to perform; and
during or subsequent to performing the adjustment of the one or more frequencies, tracking the subject using the plurality of motion trackers of the motion tracking system, wherein, during the tracking of the subject, one or more of the plurality of motion trackers is disposed on or adjacent to the one or more body members of the subject.

2. The method of claim 1, wherein the one or more frequencies is adjusted by one or more corresponding adjustment values based at least in part on a body member of the one or more body members associated with the one or more motion trackers.

3. The method of claim 1, wherein the adjustment of the one or more frequencies comprises adjusting the one or more frequencies of each of the plurality of motion trackers.

4. The method of claim 1, wherein the adjustment of the one or more frequencies comprises adjusting the one or more frequencies based at least in part on a level of motion associated with the subject, as determined by a previously recorded measurement of the subject by the motion tracking system.

5. The method of claim 1, wherein the tracking of the subject is performed during the adjustment of the one or more frequencies.

6. The method of claim 5, further comprising, subsequent to the adjustment of the one or more frequencies, performing an additional adjustment of the one or more frequencies of one or more of the plurality of motion trackers during the performance of the movement by the subject to further reduce the power consumption of the at least one of the plurality of motion trackers.

7. The method of claim 6, wherein the additional adjustment is based at least in part on a measured level of motion of the one or more motion trackers.

8. The method of claim 7, wherein a magnitude of the additional adjustment is based on the measured level of motion and a range of motion associated with the movement which the subject is instructed to perform.

9. The method of claim 1, further comprising adjusting a transmission frequency of at least one of the plurality of motion trackers such that substantially no transmission occurs when the at least one of the plurality of motion trackers is motionless.

10. The method of claim 9, further comprising:
detecting, by the motion tracking system, that the at least one of the plurality of motion trackers is motionless;
determining that the at least one of the plurality of motion trackers is not expected to be motionless within a tracked motion range associated with the movement which the subject is instructed to perform;
halting tracking of the movement; and
providing an alert of a fault condition of the motion tracking system.

11. The method of claim 1, wherein the plurality of motion trackers each comprises an inertial measurement unit.

12. The method of claim 11, wherein the inertial measurement unit comprises at least two of an accelerometer, a gyroscope, and a magnetometer.

13. The method of claim 1, wherein at least one of the plurality of motion trackers comprises a vital sign sensor.

14. The method of claim 13, wherein the vital sign sensor comprises a respiration rate sensor, a body temperature sensor, a pulse rate sensor, or a combination of two or more thereof.

15. The method of claim 14, wherein the one or more frequencies are repeatedly adjusted during operation of the motion tracking system.

16. The method of claim 15, wherein the one or more frequencies are repeatedly adjusted to maintain at least a threshold motion tracking accuracy level while increasing a life of a battery of each of the plurality of motion trackers.

17. The method of claim 16, wherein a hardware configuration of each of the plurality of motion trackers is identical.

18. The method of claim 1, wherein each of the plurality of motion trackers is powered by a battery.

19. A motion tracking system comprising at least one computing device and a plurality of motion trackers in communication with the at least one computing device, and configured to perform operations comprising:
using a user interface provided by the computing device to instruct a subject to perform a movement of one or more body members of the subject;
performing an adjustment of one or more frequencies of one or more of the plurality of motion trackers to reduce a power consumption of at least one of the plurality of motion tracker to thereby reduce a power consumption of the motion tracking system, wherein the one or more frequencies is adjusted based at least in part on the movement which the subject is instructed to perform; and
during or subsequent to performing the adjustment of the one or more frequencies, tracking the subject using the plurality of motion trackers of the motion tracking system, wherein, during the tracking of the subject, one or more of the plurality of motion trackers is disposed on or adjacent to the one or more body members of the subject.

20. A non-transitory computer readable medium comprising instructions that, when executed by at least one computer processor, cause the at least one computer processor to perform operations comprising:
in a motion tracking system comprising a computing device and a plurality of motion trackers in communication with the computing device, using a user interface provided by the computing device to instruct a subject to perform a movement of one or more body members of the subject;
performing an adjustment of one or more frequencies of one or more of the plurality of motion trackers to reduce a power consumption of at least one of the plurality of motion tracker to thereby reduce a power consumption of the motion tracking system, wherein the one or more frequencies is adjusted based at least in part on the movement which the subject is instructed to perform; and
during or subsequent to performing the adjustment of the one or more frequencies, tracking the subject using the plurality of motion trackers of the motion tracking system, wherein, during the tracking of the subject, one or more of the plurality of motion trackers is disposed on or adjacent to the one or more body members of the subject.
